(19) United States
(12) Patent Application Publication
(10) Pub. No.: US 2013/0116254 A1
(43) Pub. Date: May 9, 2013

(54) COMPOSITIONS AND METHODS FOR LOWERING INTRAOCULAR PRESSURE

(71) Applicant: Chetan P. Pujara, Irvine, CA (US)

(72) Inventor: Chetan P. Pujara, Irvine, CA (US)

(73) Assignee: Allergan, Inc., Irvine, CA (US)

(21) Appl. No.: 13/698,182

(22) PCT Filed: Dec. 21, 2010
(86) PCT No.: PCT/US10/61563

§ 371 Date: Nov. 15, 2012

Related U.S. Application Data
(60) Provisional application No. 61/288,936, filed on Dec. 22, 2009.
(60) Provisional application No. 61/361,749, filed on Jul. 6, 2010.

(51) Int. Cl. A61K 31/5377; A61K 31/5375

(52) U.S. Cl. 514/236.2

(57) ABSTRACT

Disclosed herein are compositions for lowering intraocular pressure (IOP) of an eye comprising a combination of the IOP-lowering agents bimatoprost, brimonidine, and timolol. Further disclosed are methods for reducing IOP in the eye of a subject.

### CROSS REFERENCE TO RELATED APPLICATION

This application claims priority to U.S. Provisional Application No. 61/288,936, filed Dec. 22, 2009 and U.S. Provisional Application No. 61/361,749 filed Jul. 6, 2010 which are incorporated herein by reference in their entirety.

### FIELD

Embodiments disclosed herein provide compositions and methods that lower intraocular pressure. The compositions and methods disclosed herein include bimatoprost, brimonidine and timolol and combinations thereof and are particularly suited for patients who require maximum medical therapy for lowering intraocular pressure and for treatment of glaucoma.

### BACKGROUND

Numerous disturbances or disorders of the eye lead to an increase in intraocular pressure (IOP). For example, post-surgical or post-laser trabeculectomy, ocular hypertensive episodes and glaucoma all can result in increased IOP.

On the basis of its etiology, glaucoma has been classified as primary or secondary. Primary glaucoma, also known as congenital glaucoma, can occur in the absence of other ocular conditions. The underlying causes of primary glaucoma are not known. It is known, however, that the increased IOP observed in primary glaucoma is due to the obstruction of aqueous humor flow out of the eye. In chronic open-angle primary glaucoma, the anterior chamber and its anatomic structures appear normal, but drainage of the aqueous humor is impeded. In acute or chronic angle-closure primary glaucoma, the anterior chamber is shallow, the filtration angle is narrowed, and the iris may obstruct the trabecular meshwork at the entrance of the canal of Schlemm. Dilation of the pupil may also push the root of the iris forward against the angle to produce pupillary block precipitating an acute attack. Additionally, eyes with narrow anterior chamber angles are predisposed to acute angle-closure glaucoma attacks of various degrees of severity.

Secondary glaucoma results from another pre-existing ocular disease such as, without limitation, uveitis, intraocular tumor, enlarged cataract, central retinal vein occlusion, trauma to the eye, operative procedures and intraocular hemorrhage. Accordingly, any interference with the outward flow of aqueous humor from the posterior chamber into the anterior chamber and subsequently, into the canal of Schlemm can lead to secondary glaucoma.

Considering all types of glaucoma together, this ocular disorder occurs in about 2% of all persons over the age of 40. Unfortunately, glaucoma can be asymptomatic for years before progressing to a rapid loss of vision.

In cases where surgery is not indicated, topical β-adrenoreceptor antagonists have traditionally been the drugs of choice for treating glaucoma. Certain eicosanoids and their derivatives have also been reported to possess ocular hypotensive activity, and have been recommended for use in glaucoma management. Eicosanoids and their derivatives include numerous biologically important compounds such as prostaglandins and their derivatives. While prostaglandins were earlier regarded as potent ocular hypertensives, evidence has accumulated that some prostaglandins are highly effective ocular hypotensive agents ideally suited for long-term medical management of glaucoma.

Prostaglandins can be described as derivatives of prostanoic acid which have the structural formula:

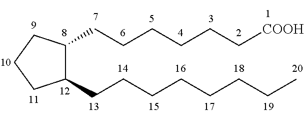

Particularly useful hypotensive prostaglandins include PGF_{2α}, PGF_{1α}, PGE_{2α}, and certain lipid-soluble esters, such as C₁ to C₅ alkyl esters, e.g. 1-isopropyl ester, of such compounds. Many patients needing to lower their intraocular pressure are on fixed combination therapies such as COMBIGAN® and GANFORT®. However, for some patients, combination therapies are not enough to lower intraocular pressure and triple combination therapy is required. The combined effect is expected to result potentially in additional IOP reduction in patients with chronic open-angle glaucoma or ocular hypertension who are not well controlled on 2 IOP-lowering agents.

### SUMMARY

Embodiments disclosed herein relate to enhanced medical therapy for patients with increased intraocular pressure (IOP) using a combination of IOP-lowering agents. Particularly, embodiments disclosed herein contain a triple combination of IOP-lowering agents for use by patients with increased IOP providing superior efficacy while maintaining safety and tolerability. In certain embodiments, the patient or subject is human.

Bimatoprost is a potent ocular hypotensive agent (Cantor, 2001; Sherwood et al, 2001). It is a synthetic prostamide, structurally related to prostaglandin F2α (PGF2α), that selectively mimics the effects of biosynthesized substances called prostamides. Bimatoprost reduces IOP in humans by increasing aqueous humor outflow through the trabecular meshwork and enhancing uveoscleral outflow (Brubaker et al, 2001). Brimonidine tartrate is an alpha-2 adrenergic receptor agonist that is 1000-fold more selective for the alpha-2 adrenoceptor than the alpha-1 adrenoreceptor (Munk et al, 1994). It is thought that brimonidine tartrate lowers IOP by enhancing uveoscleral outflow and reducing aqueous humor formation (Report 610-94-012; Serle et al, 1991). Timolol is a beta-1 and beta-2 non-selective adrenergic receptor blocking agent. Timolol lowers IOP by reducing aqueous humor formation (Coakes and Brubaker, 1978; Yablonski et al, 1978).

According to the various example embodiments, the compositions contain bimatoprost, brimonidine, and timolol. In another embodiment, the brimonidine is a salt thereof, such as brimonidine tartrate, and the timolol is a salt thereof, such as timolol maleate. In another embodiment, the compositions further contain sodium phosphate dibasic heptahydrate, citric acid monohydrate, sodium chloride, and sodium hydroxide in an aqueous carrier. In yet another embodiment, the compositions further contain benzalkonium chloride.

In another embodiment, the compositions contain 0.01% w/v bimatoprost, 0.15% w/v brimonidine tartrate, and 0.683% w/v timolol maleate. In this combination, both brimonidine tartrate and timolol maleate are at concentrations and regimens that are approved for the individual components. Bimatoprost in this combination, however, is at 0.01%, which is lower than the 0.03% concentration approved.

In yet another embodiment, the compositions further contain 1.5% w/v sodium phosphate dibasic; heptahydrate, 0.025% w/v citric acid monohydrate, and 0.35% w/v sodium chloride. In yet another embodiment, the compositions further contain 0.005% w/v benzalkonium chloride.

In another embodiment, the compositions consist essentially of bimatoprost, brimonidine, and timolol. In another embodiment, the brimonidine is a salt thereof, such as brimonidine tartrate, and the timolol is a salt thereof, such as timolol maleate. In yet another embodiment, the compositions further consist essentially of sodium phosphate dibasic heptahydrate, citric acid monohydrate, sodium chloride, and sodium hydroxide in an aqueous carrier. In yet another embodiment, the compositions further consist essentially of benzalkonium chloride.

In another embodiment, the compositions consist essentially of 0.01% w/v bimatoprost, 0.15% w/v brimonidine tartrate, and 0.68% w/v timolol maleate. In another embodiment, the compositions further consist essentially of 1.5% w/v sodium phosphate dibasic heptahydrate, 0.025% w/v citric acid monohydrate, 0.35% w/v sodium chloride, sodium hydroxide, in an aqueous carrier. In yet another embodiment, the compositions further consists essentially of 0.005% w/v benzalkonium chloride.

In another embodiment, the compositions consist of bimatoprost, brimonidine, timolol, sodium phosphate dibasic heptahydrate, citric acid monohydrate, sodium chloride, and sodium hydroxide in an aqueous carrier. In another embodiment, the brimonidine is a salt thereof, brimonidine tartrate, and the timolol is a salt thereof, timolol maleate. In yet another embodiment, the compositions further consist of benzalkonium chloride.

In another embodiment, the compositions consist of 0.01% w/v bimatoprost, 0.15% w/v brimonidine tartrate, and 0.68% w/v timolol maleate, 1.5% w/v sodium phosphate dibasic heptahydrate, 0.025% w/v citric acid monohydrate, 0.35% w/v sodium chloride, sodium hydroxide, and water. In yet another embodiment, the compositions further consist of 0.005% w/v benzalkonium chloride.

In certain embodiments disclosed herein, the compositions do not contain, consist of, or consist essentially of components other than bimatoprost, brimonidine, timolol, sodium phosphate dibasic heptahydrate, citric acid monohydrate, sodium chloride, benzalkonium chloride, and sodium hydroxide in an aqueous carrier.

Embodiments disclosed herein also include methods of reducing IOP through the administration of compositions containing bimatoprost, brimonidine, and timolol. In another embodiment the brimonidine is a salt thereof, such as brimonidine tartrate, and the timolol is a salt thereof, such as timolol maleate. In yet another embodiment the administered compositions further contain sodium phosphate dibasic heptahydrate, citric acid monohydrate, sodium chloride, and sodium hydroxide in, an aqueous carrier. In yet another embodiment the administered compositions further contain benzalkonium chloride.

In another embodiment the method of lowering IOP includes administering compositions containing 0.01% w/v bimatoprost, 0.15% w/v brimonidine tartrate, and 0.68% w/v timolol maleate. In yet another embodiment, the administered compositions further contain 1.5% w/v sodium phosphate dibasic heptahydrate, 0.025% w/v citric acid monohydrate, 0.35% w/v sodium chloride, sodium hydroxide, in an aqueous carrier. In yet another embodiment, the administered compositions further contain 0.005% w/v benzalkonium chloride.

Embodiments disclosed herein also include methods of reducing IOP through the administration of compositions described herein to subjects or patients. In certain embodiments the compositions described herein are administered to human subjects.

Embodiments, disclosed herein also include methods of reducing IOP through the administration of compositions that consist essentially of bimatoprost, brimonidine, and timolol. In another embodiment the brimonidine is a salt thereof, such as brimonidine tartrate, and the timolol is a salt thereof, such as timolol maleate. In yet another embodiment the administered compositions further consist essentially of sodium phosphate dibasic heptahydrate, citric acid monohydrate, sodium chloride, and sodium hydroxide in an aqueous carrier. In yet another embodiment the administered compositions further consist essentially of benzalkonium chloride.

In another embodiment the method of lowering IOP includes administering compositions consisting essentially of 0.01% w/v bimatoprost, 0.15% w/v brimonidine tartrate, and 0.68% w/v timolol maleate. In yet another embodiment, the administered compositions further consist essentially of 1.5% w/v sodium phosphate dibasic heptahydrate, 0.025% w/v citric acid monohydrate, 0.35% w/v sodium chloride, and sodium hydroxide in an aqueous carrier. In yet another embodiment, the administered compositions further consist essentially of 0.005% w/v benzalkonium chloride.

Embodiments disclosed herein also include methods of reducing IOP through the administration of compositions that consist of bimatoprost, brimonidine, timolol, sodium phosphate dibasic heptahydrate, citric acid monohydrate, sodium chloride, and sodium hydroxide in an aqueous carrier. In another embodiment the brimonidine is the salt, brimonidine tartrate, and the timolol is the salt, timolol maleate. In yet another embodiment the administered compositions further consist of benzalkonium chloride.

In another embodiment the method of lowering IOP includes administering compositions consisting of 0.01% w/v bimatoprost, 0.15% w/v brimonidine tartrate, 0.68% w/v timolol maleate, 1.5% w/v sodium phosphate dibasic heptahydrate, 0.025% w/v citric acid monohydrate, 0.35% w/v sodium chloride, and sodium hydroxide in an aqueous carrier. In yet another embodiment, the administered compositions further consist of 0.005% w/v benzalkonium chloride.

### DEFINITION OF TERMS

For the purposes of this disclosure, “treat,” “treating,” “treatment,” or “therapy” refer to the use of a compound, composition, therapeutically active agent, or drug in the diagnosis, cure, mitigation, or treatment of the disease or underlying condition.

A “pharmaceutically acceptable salt” is any salt that retains the activity of the parent compound and does not impart any additional deleterious or untoward effects on the subject to which it is administered and in the context in which it is administered compared to the parent compound. A pharmaceutically acceptable salt also refers to any salt which can form in vivo as a result of administration of an acid, another salt, or a prodrug which is converted into an acid or salt.

Pharmaceutically acceptable salts of acidic functional groups can be derived from organic or inorganic bases. The salt can comprise a mono or polyvalent ion. Of particular interest are the inorganic ions lithium, sodium, potassium, calcium, and magnesium. Organic salts can be made with amines, particularly ammonium salts such as mono-, di- and trialkyl amines or ethanol amines. Salts can also be formed with caffeine, tromethamine and similar molecules. Hydrochloric acid or some other pharmaceutically acceptable acid can form a salt with a compound that includes a basic group, such as an amine or a pyridine ring.

A “prodrug” is a compound which is converted to a therapeutically active compound after administration, and the term should be interpreted as broadly herein as is generally understood by one of ordinary skill in the art. While not intending to limit the scope of this disclosure, conversion can occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester can be derived from a carboxylic acid of Cl (i.e. the terminal carboxylic acid of a natural prostaglandin), or an ester can be derived from a carboxylic acid functional group on another part of the molecule, such as on a phenyl ring. While not intending to be limiting, an ester can be an alkyl ester, an aryl ester, or a heteroaryl ester. The term alkyl has the meaning generally understood by those of ordinary skill in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

### DESCRIPTION

In a series of United States patent applications assigned to Allergan, Inc., prostaglandin esters with increased ocular hypotensive activity accompanied with substantially reduced side-effects are disclosed. U.S. patent application Ser. No. 386,835 (filed Jul. 27, 1989), relates to certain 11-acyl-prostaglandins, such as 11-pivaloyl, 11-acetyl, 11-isobutyryl, 11-valeryl, and 11-isovaleryl PGF_{2α} useful for lowering intraocular pressure (IOP). Intraocular pressure-reducing 15-acyl prostaglandins are disclosed in U.S. patent application Ser. No. 357,394 (filed May 25, 1989). Similarly, 11,15-9,15- and 9,11-diesters of prostaglandins, for example 11,15-dipivaloyl PGF_{2α} have ocular hypotensive activity. See U.S. patent application Ser. No. 385,645 (filed Jul. 27, 1990), now U.S. Pat. No. 4,994,274; U.S. patent application Ser. No. 584,370 (filed Sept. 18, 1990), now U.S. Pat. No. 5,028,624, which is a continuation of U.S. patent application Ser. No. 386,312 (filed Jul. 27, 1989), and U.S. patent application Ser. No. 585,284 (filed Sept. 18, 1990), now U.S. Pat. No. 5,034,413 which is a continuation of U.S. patent application Ser. No. 385,834 (filed Jul. 27, 1989). Each of these references is incorporated by reference herein in its entirety for its teachings regarding prostaglandin esters with ocular hypotensive activity.

Disclosed herein are compositions and methods for lowering IOP using a combination of at least three IOP-lowering agents, or pharmaceutical salts or prodrugs thereof including bimatoprost, brimonidine, and timolol. In certain embodiments, additional ingredients are added to the triple combination of bimatoprost, brimonidine, and timolol to make the composition more ophthalmically acceptable, including, without limitation, preservatives, buffers, tonicity adjusters, and surfactants. Additionally, various vehicles can be used in the disclosed embodiments. These compositions are useful in reducing IOP in patients with increased IOP, thus, for example, preventing or delaying glaucoma in those with ocular hypertension, and preventing or delaying further vision loss in those with glaucoma.

Without wishing to be bound by any particular theory, it is thought that the prostamide analog, bimatoprost (sold by Allergan, Inc. under the name LUMIGAN®) reduces IOP by increasing the aqueous humor outflow of an eye. Bimatoprost's chemical name is (Z)-7-[(1R,2R,3R,5S)-3,5-Dihydroxy-2-[1E,3S)-3-hydroxy-5-phenyl-1-pentenyl]cyclopentyl]-5-N-ethylheptenamide, and it has a molecular weight of 415.58.

Brimonidine, an α2-andrenergic agonist receptor, reduces the body's production of aqueous humor and increases the flow of aqueous humor out of the eye, resulting in a decrease in IOP. Brimonidine is available from Allergan, Inc. as ALPAHAGAN®. The chemical name of brimonidine tartrate, a salt of brimonidine, is 5-bromo-6-(2-imidazolidinylideneamino) quinoxaline L-tartrate. Brimonidine has a molecular weight of 442.24 as the tartrate salt.

Timolol, a non-selective β-adrenergic receptor blocking agent, reduces the body's aqueous humor production through the blockage of the β receptors on the ciliary epithelium. In one embodiment, the timolol component contains an acid salt of timolol and in another embodiment contains timolol maleate. The chemical name of timolol maleate, is (+1-tert-butylamino)-3-[(4-morpholino-1,2,5-thiodiazol-3yl) oxy]-2-propanol maleate (1:1) (salt). Timolol maleate has a molecular weight of 432.50. Timolol is commercially available from Merck as TIMOPTIC®.

Preservatives that can be used in the pharmaceutical compositions of the present embodiments include, but are not limited to, benzalkonium chloride, chlorobutanol, thimerosal, phenylmercuric acetate and phenylmercuric nitrate. Preservative-free compositions can be considered, in one non-limiting embodiment for patients experiencing hypersensitivity reactions with the above listed preservatives or other preservatives not listed.

Various buffers and means for adjusting pH can be used so long as the resulting preparation is ophthalmically acceptable. Accordingly, buffers include acetate buffers, citrate buffers, phosphate buffers and borate buffers. Acids or bases can also be used to adjust the pH of these formulations as needed. The pH of the disclosed compositions should preferably be maintained between 6.5 and 7.2 with an appropriate buffer system.

Tonicity adjustors can be added as needed and include, without limitation, glycerin, sorbitol, sodium chloride, potassium chloride, and mannitol, or any other suitable ophthalmically acceptable tonicity adjustor. In one embodiment the tonicity adjustor is sodium chloride.

In certain embodiments, a surfactant such as a polysorbate, for example, a TWEEN® by Sigma, can be added. Further, any other suitable surfactants can be used as well.

Various vehicles can also be used in the ophthalmic preparations of the present embodiments. These vehicles include, but are not limited to, polyvinyl alcohol, povidone, hydroxypropyl methyl cellulose, poloxamers, carboxymethyl cellulose, hydroxyethyl cellulose, physiological saline solution, water, purified water, and combinations thereof.

Additionally, ophthalmically acceptable antioxidants can be included in the disclosed compositions. Suitable antioxidants include, but are not limited to, sodium metabisulfite, sodium thiosulfate, acetylcysteine, butylated hydroxyanisole and butylated hydroxytoluene and the like and mixtures thereof.

Another excipient component that can be included in the ophthalmic preparations are chelating agents. A useful chelating agent is edetate disodium, although other chelating agents can also be used in place of or in conjunction edetate disodium.

Compositions and methods disclosed herein can also be used in combination with the following classes of drugs, pharmaceutically acceptable salts or prodrugs thereof:

β—Blockers (or β-adrenergic antagonists) including, without limitation, carteolol, levobunolol, metiparanolol, timolol hemihydrate, β1-selective antagonists such as betaxolol, and the like;
Adrenergic Agonists including, without limitation, non-selective adrenergic agonists such as epinephrine borate, epinephrine hydrochloride, and dipivefrin, and the like; and α₂ -selective adrenergic agonists such as apraclonidine and the like;
Carbonic Anhydrase Inhibitors including, without limitation, acetazolamide, dichlorphenamide, methazolamide, brinzolamide, dorzolamide, and the like;
Cholinergic Agonists including, without limitation, direct acting cholinergic agonists such as carbachol, pilocarpine hydrochloride, pilocarbine nitrate, pilocarpine, and the like;
Chlolinesterase inhibitors such as demecarium, echothiophate, physostigmine, and the like;
Glutamate Antagonists and other neuroprotective agents such as Ca²⁺ channel blockers such as memantine, amantadine, rimantadine, nitroglycerin, dextrophan, detromethorphan, CGS-19755, dihydropyridines, verapamil, emopamil, benzothiazepines, bepridil, diphenylbutylpiperidines, diphenylpiperazines, HOE 166 and related drugs, fluspirilene, eliprodil, ifenprodil, CP-101,606, tibalosine, 2309BT, and 840S, flunarizine, nicardipine, nifedimpine, nimodipine, barnidipine, verapamil, lidoflazine, prenylamine lactate, amiloride, and the like;
Additional prostamides or pharmaceutically acceptable salts or prodrugs thereof;
Prostaglandins including travoprost, UFO-21, chloprostenol, fluprostenol, 13,14-dihydro-chloprostenol, isopropyl unoprostone, latanoprost and the like; and
Cannabinoids including CB1 agonists such as WIN-55212-2 and CP-55940 and the like.

For treatment of diseases affecting the eye, the disclosed compositions can be administered topically or as ocular implants.

Pharmaceutical compositions can be prepared by combining a therapeutically effective amount of bimatoprost, brimonidine and timolol according to the present disclosure, or pharmaceutically acceptable acid addition salts thereof, as active ingredients, with conventional ophthalmically acceptable pharmaceutical excipients, and by preparation of unit dosage forms suitable for ocular use. The therapeutically efficient amount will vary with the activity of the active ingredients; however, typically in combination will be between 0.0001 and 20% (w/v), between 0.0001 and 10% (w/v), between 0.0001 and 5% (w/v), between 0.0005 and 3% (w/v), between 0.00075 and 2% (w/v), between 0.001 and 1.0% (w/v), between 0.2 and 1.0% (w/v), between 0.5 and 1.0%(w/v), 0.85%(w/v) or 0.843% (w/v) of the composition. The compositions can be prepared as follows:
1. Add quantity of water i.e. approximately 70% of the batch size in a chosen stainless steel vessel.
2. Add Sodium Phosphate Dibasic Heptahydrate to step 1 under mechanical stirring and mix until dissolved.
3. Add Citric Acid Monohydrate to step 2 under stirring and mix until dissolved.
4. Add Sodium Chloride to step 3 under stirring and mix until dissolved.
5. Add bimatoprost to step 4 under stirring and mix until a clear solution is obtained.
6. Add timolol maleate to step 5 under stirring and mix until dissolved.
7. Add brimonidine tartrate to step 6 under stirring and mix until dissolved.
8. Add benzalkonium chloride to step 7 as a stock solution under stirring.
9. Check pH of the solution, adjust if necessary to pH 7.1
10. Make up the volume to 100% of the batch size with water and stir for 5-10 minutes.

Bimatoprost can be included in compositions of the embodiments disclosed herein in an amount of between 0.0001 and 15% (w/v), between 0.0001 and 10% (w/v), between 0.0001 and 5% (w/v), between 0.0005 and 3% (w/v), between 0.00075 and 2% (w/v), between 0.001 and 1.0% (w/v), between 0.001 and 0.1 (w/v), between 0.005 and 0.05%(w/v), or 0.01% (w/v) of the composition.

Brimonidine can be included in compositions of the embodiments disclosed herein in an amount of between 0.0001 and 15% (w/v), between 0.0001 and 10% (w/v), between 0.0001 and 5% (w/v), between 0.0005 and 3% (w/v), between 0.00075 and 2% (w/v), between 0.001 and 1.0% (w/v), between 0.001 and 0.2 (w/v), between 0.005 and 0.05%(w/v), or 0.15% (w/v) of the composition. In one embodiment brimonidine is provided as brimonidine tartrate in an amount of 0.15% (w/v) of the composition.

Timolol can be included in compositions of the embodiments disclosed herein in an amount of between 0.0001 and 15% (w/v), between 0.0001 and 10% (w/v), between 0.0001 and 5% (w/v), between 0.0005 and 3% (w/v), between 0.01 and 2% (w/v), between 0.1 and 1.0% (w/v), between 0.1 and 0.9 (w/v), between 0.3 and 0.8% (w/v), 0.5%, 0.6% (w/v), 0.68% (w/v/) or 0.683% (w/v) of the composition. In one embodiment timolol is provided as timolol maleate in an amount of 0.6%, 0.68% or 0.683% (w/v) of the composition.

The amount of the presently useful compositions administered is dependent on the therapeutic effect or effects desired, on the specific patient being treated, on the severity and nature of the patient's condition, on the manner of administration, on the potency and pharmacodynamics of the particular compound or compounds employed, and on the judgment of the prescribing physician. The therapeutically effective dosage of the presently useful compositions can be in the range of 0.01 to 200 mg/kg/day. In certain embodiments, the therapeutically effective dosage can be 0.1, 0.5, 1, 2.5, 5, 15, 20, 25, 50, 60, 70, 75, 80, 85, 90 or 100 mg/kg/day. The dosage can be provided in a single daily dosage or in a number of doses from 2 to 24 over the course of day. In certain embodiments, dosages can be administered every other day, every third day, once a week, once a month, etc.

In preferred embodiments, the comfort of formulations disclosed herein is maximized as much as possible, although sometimes formulation considerations (e.g. drug stability) can necessitate less than optimal comfort. In the case that comfort cannot be maximized, the compositions should be formulated such that the compositions are tolerable to the patient for ophthalmic use.

The ophthalmic formulations of the present disclosure are conveniently packaged in forms suitable for metered application, such as in containers equipped with a dropper, to facilitate application to the eye. Containers suitable for dropwise application are usually made of suitable inert, non-toxic plastic material, and generally contain between 0.5 and 15 ml solution. One package can contain one or more unit doses.

Preservative-free solutions (e.g., Table 7) can be formulated in non-resealable containers containing up to 1, 2, 5, 10, 50, or 100 unit doses, where a typical unit dose is from 1 to 8 drops. The volume of one drop generally will be from 20 to 35 μl.

Various exemplary embodiments can be formulated as shown in the Tables follows:

TABLE 1

| Ingredients | Amount (% w/v) |
|---|---|
| Active Ingredients | 0.001-5 |
| Preservative | 0-0.10 |
| Vehicle | 0-40 |
| Tonicity Adjuster | 1-10 |
| Buffer | 0.01-10 |
| pH Adjuster | q.s. pH 4.5-7.5 |
| Antioxidant | As needed |
| Surfactant | As needed |
| Purified Water | As needed to make 100% |

TABLE 2

| Ingredient | Function | % w/v |
|---|---|---|
| Bimatoprost | Active | 0.003-0.03 |
| Brimonidine | Active | 0.005-0.2 |
| Timolol | Active | 0.2-0.5 |
| Sodium Phosphate | Buffering Agent | As needed to make |
| Dibasic Heptahydrate | | ophthalmically acceptable |
| Citric Acid Monohydrate | Buffering Agent | As needed to make |
| | | ophthalmically acceptable |
| Sodium Chloride | Tonicity Agent | As needed to make |
| | | ophthalmically acceptable |
| Sodium Hydroxide | pH Adjuster | q.s. |
| Water | Vehicle | q.s. |

TABLE 3

| Ingredient | Function | % w/v |
|---|---|---|
| Bimatoprost | Active | 0.003-0.03 |
| Brimonidine Tartrate | Active | 0.005-0.3 |
| Timolol Maleate | Active | 0.2-0.8 |
| Sodium Phosphate Dibasic | Buffering Agent | 1.0-2.0 |
| Heptahydrate | | |
| Citric Acid Monohydrate | Buffering Agent | 0.01-0.05 |
| Sodium Chloride | Tonicity Agent | 0.10-0.30 |
| Sodium Hydroxide | pH Adjuster | q.s. |
| Water | Vehicle | q.s. |

TABLE 4

| Ingredient | Function | % w/v |
|---|---|---|
| Bimatoprost | Active | 0.003-0.03 |
| Brimonidine | Active | 0.005-0.2 |
| Timolol | Active | 0.2-0.5 |
| Sodium Phosphate | Buffering Agent | As needed to make |
| Dibasic Heptahydrate | | ophthalmically acceptable |
| Citric Acid Monohydrate | Buffering Agent | As needed to make |
| | | ophthalmically acceptable |
| Sodium Chloride | Tonicity Agent | As needed to make |
| | | ophthalmically acceptable |
| Benzalkonium Chloride | Preservative | As needed to make |
| | | ophthalmically acceptable |
| Sodium Hydroxide | pH Adjuster | q.s. |
| Water | Vehicle | q.s. |

TABLE 5

| Ingredient | Function | % w/v |
|---|---|---|
| Bimatoprost | Active | 0.01 |
| Brimonidine Tartrate | Active | 0.1 |
| Timolol Maleate | Active | 0.6 |
| Sodium Phosphate Dibasic | Buffering Agent | 1.5 |
| Heptahydrate | | |
| Citric Acid Monohydrate | Buffering Agent | 0.025 |
| Sodium Chloride | Tonicity Agent | 0.35 |
| Sodium Hydroxide | pH Adjuster | q.s. |
| Water | Vehicle | q.s. |

TABLE 6

| Ingredient | Function | % w/v |
|---|---|---|
| Bimatoprost | Active | 0.01 |
| Brimonidine Tartrate | Active | 0.1 |
| Timolol Maleate | Active | 0.6 |
| Sodium Phosphate Dibasic | Buffering Agent | 1.5 |
| Heptahydrate | | |
| Citric Acid Monohydrate | Buffering Agent | 0.025 |
| Sodium Chloride | Tonicity Agent | 0.35 |
| Benzalkonium Chloride | Preservative | 0.005 |
| Sodium Hydroxide | pH Adjuster | q.s. |
| Water | Vehicle | q.s. |

TABLE 7

| Ingredient | Function | % w/v |
|---|---|---|
| Bimatoprost | Active | 0.01 |
| Brimonidine Tartrate | Active | 0.15 |
| Timolol Maleate | Active | 0.68 |
| Sodium Phosphate Dibasic | Buffering Agent | 1.5 |
| Heptahydrate | | |
| Citric Acid Monohydrate | Buffering Agent | 0.025 |
| Sodium Chloride | Tonicity Agent | 0.35 |
| Sodium Hydroxide | pH Adjuster | q.s. |
| Water | Vehicle | q.s. |

TABLE 8

| Ingredient | Function | % w/v |
|---|---|---|
| Bimatoprost | Active | 0.01 |
| Brimonidine Tartrate | Active | 0.15 |
| Timolol Maleate | Active | 0.68 |
| Sodium Phosphate Dibasic | Buffering Agent | 1.5 |
| Heptahydrate | | |
| Citric Acid Monohydrate | Buffering Agent | 0.025 |
| Sodium Chloride | Tonicity Agent | 0.35 |
| Benzalkonium Chloride | Preservative | 0.005 |
| Sodium Hydroxide | pH Adjuster | q.s. |
| Water | Vehicle | q.s. |

TABLE 9

| Ingredient | Function | % w/v |
|---|---|---|
| Bimatoprost | Active | 0.01 |
| Brimonidine Tartrate | Active | 0.15 |
| Timolol Maleate | Active | 0.683 |
| | | (equivalent to |
| | | 0.5 timolol) |
| Sodium Phosphate | Buffering Agent | 1.5 |
| Dibasic Heptahydrate | | |
| Citric Acid Monohydrate | Buffering Agent | 0.025 |
| Sodium Chloride | Tonicity Agent | 0.35 |
| Sodium Hydroxide | pH Adjuster | q.s. |
| Water | Vehicle | q.s. |

TABLE 10

| Ingredient | Function | % w/v |
|---|---|---|
| Bimatoprost | Active | About 0.01 |
| Brimonidine Tartrate | Active | About 0.15 |
| Timolol Maleate | Active | About 0.683 |
| Sodium Phosphate Dibasic | Buffering Agent | About 1.5 |
| Heptahydrate | | |
| Citric Acid Monohydrate | Buffering Agent | About 0.025 |
| Sodium Chloride | Tonicity Agent | About 0.35 |
| Benzalkonium Chloride | Preservative | About 0.005 |
| Sodium Hydroxide | pH Adjuster | q.s. |
| Water | Vehicle | q.s. |

*“About” refers to variations in the concentrations which would be considered bioequivalent by a regulatory agency

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

### Example I

### Triple Combination of Bimatoprost, Brimonidine and Timolol

Thirty-six female New Zealand White rabbits, obtained from Charles River Laboratories, Wilmington, Mass., 4 months old, and weighing from 2.96 to 3.54 kilograms at the start of dosing, were used for the study. One drop (˜30 μL/drop) of the formulation of Table 11 was administered directly onto the superior corneal surface of the left eye using a dropper bottle 2 or 4 times daily (2 or 6 hour intervals) for 1 month.

TABLE 11

| Ingredient | Function | % w/v |
|---|---|---|
| Bimatoprost | Active | 0.01 |
| Brimonidine Tartrate | Active | 0.15 |
| Timolol Maleate | Active | 0.683* |
| Sodium Phosphate Dibasic Heptahydrate | Buffering Agent | 1.5 |
| Citric Acid Monohydrate | Buffering Agent | 0.025 |
| Sodium Chloride | Tonicity Agent | 0.35 |
| Benzalkonium Chloride | Preservative | 0.005 |
| Sodium Hydroxide | pH adjuster | q.s. |
| Water | Vehicle | q.s. |

*Equivalent to 0.5% w/v timolol

The contralateral right eye served as an untreated control. Dose administration and time of dosing are manually recorded onto raw data sheets, and the worksheets serve as the official record of dosing.

The intraocular pressure (IOP) in both eyes of all rabbits was measured prior to initiation of dosing and at the end of the 1-month interim period. Future examinations will occur at the end of the 3-month treatment period and following the 1-month recovery period. IOP measurements are performed at approximately the same time each examination day. An ophthalmic topical anesthetic (eg, proparacaine HCl), is administered to the eye prior to measurements. If deemed necessary, the eye is rinsed with sterile saline at the completion of the measurement. IOP measurements are manually recorded on raw data sheets.

In conclusion, female NZW rabbits (12/group) were administered one topical ocular drop (˜30 μL/drop) of Triple Combination or Triple Combination Placebo two or four times daily in the left eye for 1 month. No drug-related effects were observed in ophthalmology, gross ocular observations, body weight, clinical observations, or food consumption. Expected, acceptable drug-related decreases in intraocular pressure were observed in rabbits given Triple Combination Ophthalmic Solution. Triple Combination Ophthalmic Solution was well-tolerated.

OS-Triple Combination Ophthalmic Solution,
50 ppm BAK, (1 drop~30 μL, 4×/day)
OD-Untreated

| Study Animal | OS | | OD | |
|---|---|---|---|---|
| Number | Baseline | Day 27 | Baseline | Day 27 |
| 350 | 9 | 9 | 9 | 9 |
| 351 | 13 | 8 | 12 | 8 |
| 352 | 11 | 8 | 11 | 9 |
| 353 | 9 | 8 | 9 | 10 |
| 354 | 10 | 8 | 11 | 9 |
| 355 | 12 | 9 | 11 | 9 |
| 356 | 11 | 9 | 13 | 9 |
| 357 | 10 | 7 | 11 | 8 |
| 358 | 12 | 9 | 11 | 9 |
| 359 | 11 | 9 | 10 | 10 |
| 360 | 10 | 9 | 11 | 10 |
| 361 | 12 | 9 | 12 | 10 |
| Mean (n = 12) | 10.8 | 8.5 | 10.9 | 9.2 |
| SD | 1.3 | 0.7 | 1.2 | 0.7 |
| % Change from | — | −21.5 | — | −16.0 |
| Baseline | | | | |
| % Change from OD | −0.8 | −7.3 | — | — |
| % Change from | 0.8 | −19.0 | −5.8 | −19.1 |
| Placebo | | | | |

OS = Left eye;
OD = Right eye;
Baseline = Day-6;
“—” = Not applicable
% change from baseline = [(mean − baseline mean) + baseline mean] × 100
% change from OD = [(mean OS − mean OD) + mean OD] × 100.

### Example II

### IOP Lowering Effect of the Triple Combination Product of Table 11

A 71 year old Caucasian male is suffering from open angle glaucoma and elevated intraocular pressure which is threatening to worsen his vision if left untreated. After three months on combination therapy, the patient's IOP was not lowered to the satisfaction of his physician. The 71 year old Caucasian begins daily dosing in both eyes of the triple combination of Table 11 and the patient is expected to experience adequate lowering of IOP that was not achievable.

### Example III

### IOP Lowering of the Triple Combination Product of Table 11 in a 64 year African American Female

A 64 year old African American female suffering from glaucoma is having difficulty lowering her IOP adequately with combination therapy product of brimonidine and timolol. After four months of combination therapy of the brimonidine and timolol product, her physician switches her to the triple combination therapy product in Table 11. After three weeks on the triple combination therapy product, the patient's IOP in both eyes is expected to be at acceptable levels.

### Example III

### Treatment of Glaucoma of a 57 Year Old Caucasian Female

A 57 year old Caucasian female suffering from open-angle glaucoma has been largely non-responsive to first monotherapy and then combination therapy for lowering IOP. She switches to twice daily dosing of the composition in Table 11 and her IOP is expected to return to normal levels. After 30 days of dosing both eyes with the formulation of Table 11, the 57 year old Caucasian female's IOP returns to normal levels as long as she continues dosing with the formulation of Table 11.

### Example IV

### Treatment of Elevated Intraocular Pressure in a 61 Year Old Asian Male

A 61 year old Asian male IOP was measured with a tonometer as being between 21.3 mmHg and 23.7 mmHg. Both monotherapy and combination therapy with various therapeutic agents known to lower IOP failed to bring the patient's IOP to acceptable levels. The patient began taking twice a day administration of the formulation of Table 11, with a single drop per eye in the morning and a single drop per eye 12 hours later in the evening. After 31 days, the patient's IOP is expected to lower to between 16.1-18.2 mmHg which is considered acceptable. After 90 days, the patient's IOP is expected to lower to between 15.5-16.8 mmHg.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (epecially in the context of the following claims) are to be construed to cover both the singular and the plural, unless otherwise indicated herein or clearly contradicted by context. *“About” refers to variations in the concentrations of excipients and active agents which would be considered bioequivalent by a regulatory agency.

Recitation of ranges of values herein is merely intended to serve as a shorthand method of referring individually to each separate value falling within the range. Unless otherwise indicated herein, each individual value is incorporated into the specification as if it were individually recited herein. All methods disclosed herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are disclosed herein, including the best mode known to the inventors for carrying out the invention. Of course, variations on these described embodiments will become apparent to those of ordinary skill in the art upon reading the foregoing description. The inventor expects one of ordinary skill in the art to employ such variations as appropriate, and the inventors intend for the invention to be practiced otherwise than specifically disclosed herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1) A pharmaceutical composition for lowering intraocular pressure in a patient suffering from elevated intraocular pressure comprising bimatoprost, brimonidine, and timolol.

2) The pharmaceutical composition of claim 1 wherein the brimonidine is brimonidine tartrate and the timolol is timolol maleate.

3) The pharmaceutical composition of claim 2 further comprising sodium phosphate dibasic heptahydrate, citric acid monohydrate, sodium chloride, and sodium hydroxide in an aqueous carrier.

4) The pharmaceutical composition of claim 3 further comprising benzalkonium chloride.

5) The pharmaceutical composition of claim 2 comprising about 0.01% w/v bimatoprost, about 0.15% w/v brimonidine tartrate and about 0.68% w/v timolol maleate.

6) The pharmaceutical composition of claim 5 further comprising about 1.5% w/v sodium phosphate dibasic heptahydrate, about 0.025% w/v citric acid monohydrate and about 0.35% w/v sodium chloride.

7) The pharmaceutical composition of claim 6 further comprising about 0.005% w/v benzalkonium chloride.

8) A pharmaceutical composition consisting essentially of bimatoprost, brimonidine tartrate, timolol maleate, sodium phosphate dibasic heptahydrate, citric acid monohydrate, sodium chloride, and sodium hydroxide in an aqueous carrier.

9) The pharmaceutical composition of claim 8 further consisting essentially of benzalkonium chloride.

10) The pharmaceutical composition of claim 8 consisting essentially of 0.01% w/v bimatoprost, 0.15% w/v brimonidine tartrate, 0.68% w/v timolol maleate, 1.5% w/v sodium phosphate dibasic heptahydrate, 0.025% w/v citric acid monohydrate and 0.35% w/v sodium chloride.

11) The pharmaceutical composition of claim 10 consisting essentially of 0.005% w/v benzalkonium chloride.

12) The pharmaceutical composition of claim 8 wherein said composition consists of bimatoprost, brimonidine tartrate, timolol maleate, sodium phosphate dibasic heptahydrate, citric acid monohydrate, sodium chloride, and sodium hydroxide in an aqueous carrier.

13) The pharmaceutical composition of claim 12 further consisting of benzalkonium chloride.

14) A method of reducing intraocular pressure (IOP) in a patient suffering from elevated intraocular pressure comprising:
administering a pharmaceutical composition comprising bimatoprost, brimonidine, and timolol to the eye of a subject in need thereof.

15) The method of claim 14 wherein the pharmaceutical composition further comprises sodium phosphate dibasic heptahydrate, citric acid monohydrate, sodium chloride, and sodium hydroxide in an aqueous carrier.

16) The method of claim 15 wherein the pharmaceutical composition further comprises benzalkonium chloride and is applied to the eye at least once a day.

17) The method of claim 15 wherein said composition consists essentially of bimatoprost, brimonidine tartrate, timolol maleate, sodium phosphate dibasic heptahydrate, citric acid monohydrate, sodium chloride, and sodium hydroxide in an aqueous carrier.

18) The method of claim 17 wherein said composition further consists essentially of benzalkonium chloride.

19) The method of claim 17 wherein said composition consists essentially of 0.01% w/v bimatoprost, 0.15 % w/v brimonidine tartrate, 0.68% w/v timolol maleate, 1.5% w/v sodium phosphate dibasic heptahydrate, 0.025% w/v citric acid monohydrate and 0.35% w/v sodium chloride.

20) The method of claim 18 wherein said composition consists essentially of 0.005% w/v benzalkonium chloride.
